(19) United States
(12) Patent Application Publication
(10) Pub. No.: US 2007/0098798 A1
(43) Pub. Date: May 3, 2007

(54) Method and article to restore function to a flow control location in a patient

(75) Inventors: Yelena G. Tropsha, Plymouth, MN (US); Warren L. Starkebaum, Plymouth, MN (US)

Correspondence Address: IPLM GROUP, P.A., POST OFFICE BOX 18455, MINNEAPOLIS, MN, 55418, US

(73) Assignee: Medtronic, Inc.

(21) Appl. No.: 11/262,486

(22) Filed: Oct. 28, 2005

(51) Int. Cl. A61K 35/12; A61K 9/14

(52) U.S. Cl. 424/486; 424/93.7

(57) ABSTRACT

Article and method of restoring function to a flow control location in a patient. A biological agent is inserted into a region near the flow control location. The biological agent is stabilized in the region. The biological agent is a biological agent selected from a group consisting of growth factors, fibroblast cells, smooth muscle cells, endothelial cells, stem cells and combinations thereof.

### RELATED APPLICATION

This disclosure is related to the following co-pending application entitled “Method and Article to Restore Function to GI or Urinary Tract of a Patient” by inventor Warren L. Starkebaum (Attorney Docket No. 151P21313USO1), filed on even date herewith, which is not admitted as prior art with respect to the present disclosure by its mention in this section.

### FIELD OF THE INVENTION

The present invention is related to implantable medical devices and methods and, more particularly, to implantable medical devices and methods related to restoring function to the body of a patient.

### BACKGROUND OF THE INVENTION

Various organs of the body control the flow of bodily substances, including fluids, associated with that organ. Such organs may be located, for example, in the gastro-intestinal location, related to urinary or anal functions or in a vascular location. Examples of such locations include the upper esophageal sphincter, lower esophageal sphincter, pylorus, urinary sphincter and anal sphincter.

Any of such flow control organs may cease to function, or not function optimally, due, for example, to disease, injury and/or aging.

Lack of adequate flow control by such organs could result in discomfort, incontinence and even debilitation.

### BRIEF SUMMARY OF THE INVENTION

The present invention may allow the function of such flow control organ or organs to be restored.

In general, a biological agent may be administered to or near such flow control organ that can strengthen the tissue of the organ, enabling the organ to regain some or all of its function.

The biological agent can be stabilized at or near the location of the flow control organ to be treated. Being stabilized at or near the location of the organ, the biological agent may grow in-situ to replace or augment natural smooth muscle tissue of the organ that has been damaged or rendered non-responsive.

In an embodiment, the present invention provides a method of restoring function to a flow control location in a patient. A biological agent is inserted into a region near the flow control location. The biological agent is stabilized in the region. The biological agent is a biological agent selected from a group consisting of growth factors, fibroblast cells, smooth muscle cells, endothelial cells, stem cells and combinations thereof.

In an embodiment, the biological agent is injected into the region.

In an embodiment, the biological agent is associated with a carrier.

In another embodiment, the present invention provides an article intended to restore function to a flow control location in a patient. A carrier is adapted to be inserted into a region near the flow control location. A biological agent, associated with the carrier, selected from a group consisting of growth factors, fibroblast cells, smooth muscle cells, endothelial cells, stem cells and combinations thereof.

In an embodiment, the carrier is adapted to be inserted into tissue in the region.

In an embodiment, the carrier is a polymer matrix.

In an embodiment, the polymer matrix is a non-biodegradable polymer matrix.

In an embodiment, the polymer matrix is a biodegradable polymer matrix.

In an embodiment, the carrier is a hydrogel.

In an embodiment, the carrier is a dehydrated hydrogel.

In an embodiment, the hydrogel is allowed to hydrate following insertion.

In an embodiment, the region is selected from a group consisting of a gastro-intestinal location, a urinary location and a vascular location.

In an embodiment, the region is a location selected from the group consisting of an upper esophageal sphincter, lower esophageal sphincter, pylorus, urinary sphincter and anal sphincter.

In an embodiment, the biological agent is stabilized by placing the biological agent into tissue near the region.

In an embodiment, the tissue is muscle tissue.

### BRIEF DESCRIPTION OF THE DRAWINGS

FIG. 1 is a flow chart illustrating a method according to an embodiment of the invention;

FIG. 2 is a flow chart illustrating a method according to an alternative embodiment of the present invention;

FIG. 3 illustrates a biological agent associated with a polymer matrix carrier; and

FIG. 4 illustrates a biological agent associated with a dehydrated hydrogel.

### DETAILED DESCRIPTION OF THE INVENTION

Flow control organs in the body may be partially or fully restored to function with the stabilization of a biological agent at or near the location of the flow control organ. A flow control organ may be related the gastro-intestinal functions, urinary functions, anal functions or vascular functions. Examples of such flow control organs include the upper esophageal sphincter, lower esophageal sphincter, pylorus, urinary sphincter and anal sphincter.

The function of such a flow control organ may be partially or fully restored with the administration of a biological agent. Biological agents which may be utilized for this purpose include, but are not limited to, growth factors, fibroblast cells, smooth muscle cells, endothelial cells and stem cells, or combinations thereof.

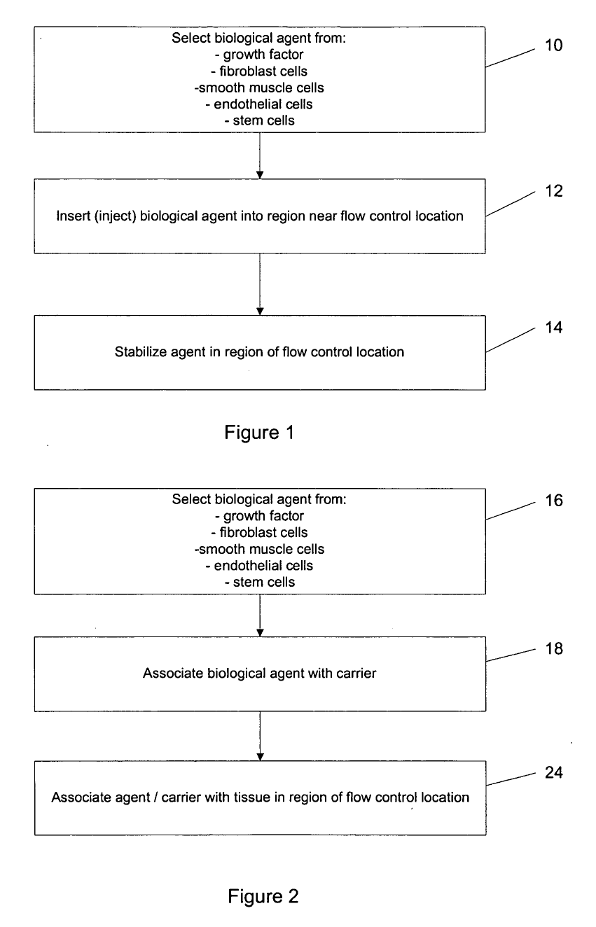

As indicated in the flow chart of FIG. 1, the biological agent may be selected (10) from several among the several classes of agents, including, but not limited to, those suggested above.

The biological agent is then inserted (12) into a region near the location of the flow control organ. The location near the flow control organ may, in fact, be at the location or in the flow control organ itself. However, beneficial results may be obtained even if the insertion location is not actually the flow control organ but rather in a location nearby. By “nearby” it is meant a location in the vicinity of the flow control organ in which the biological agent provides therapeutic effect to the flow control organ. The exact nearness depends, at least in part, on the flow control organ to be restored. Insertion of the biological agent can be accomplished by any number of conventional insertion techniques such as, for example, endoscope.

Once the biological agent has been inserted, the biological agent is stabilized (14) at the desired location. Stabilization of the biological agent can be accomplished by any number of conventional techniques. Examples of stabilization techniques that can be used are described in U.S. Patent Application Publication No. 2005/0096497, Gerber et al, Implantable Devices and Methods For Treating Fecal Incontinence, and U.S. Patent Application Publication No. 2005/0096751, Gerber et al, Implantable Devices and Methods For Treating Urinary Incontinence. Other techniques that can be used are described in U.S. Pat. No. 6,754,536, Swoyer et al, Implantable Medical Device Affixed Internally Within the Gastrointestinal Tract; U.S. Pat. No. 6,952,613, Swoyer et al, Implantable Gastrointestinal Lead With Active Fixation; U.S. Patent Application Publication No. 2002/0103521, Implantable Gastrointestinal Lead With Active Fixation; and U.S. Patent Application Publication No. 2002/0103522, Swoyer et al, Implantable Bifurcated Gastrointestinal Lead With Active Fixation. The contents of all of these documents are hereby incorporated by reference in their entirety.

FIG. 2 is a flow chart illustrating an alternative embodiment of a method of utilizing a biological agent to restore function to a flow control organ. Again biological agents which may be utilized for this purpose include, but are not limited to, growth factors, fibroblast cells, smooth muscle cells, endothelial cells and stem cells, or combinations thereof. One or more of these biological agents, or another biological agent, or a combination is selected (16) for use in the restoration process.

The biological agent is then associated (18) with a carrier. A carrier is a structure which acts to carry or harbor the biological agent and facilitate the delivery and stabilization of the biological agent to and in the location at or near the flow control organ.

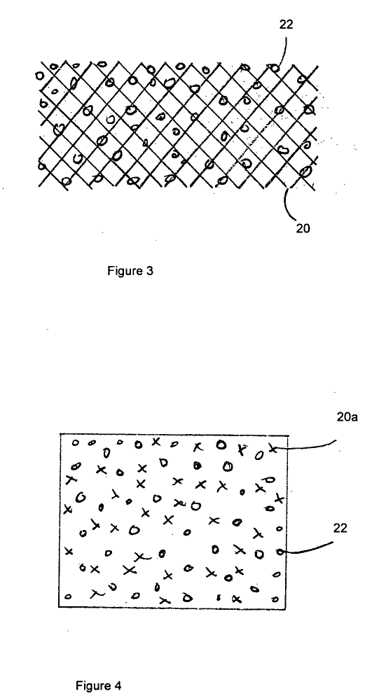

Carrier 20 illustrated in FIG. 3 is an example of a carrier that could be used for this purpose. Carrier 20 consists of a polymer matrix, preferably a biodegradable polymer matrix.

Biological agent 22 is associated with carrier 20 by, for example, saturating or impregnating carrier 20 with biological agent 22. Carrier 20 provides a physical carrier for biological agent 22 and allows biological agent 22 to be inserted into the body at the desired location at or near the flow control organ to be restored.

In a preferred embodiment illustrated in FIG. 4, carrier 20a consists of hydrogel. Carrier 20a may be filled or otherwise combined with biological agent 22. Carrier 20a and biological agent 22 combination may then be inserted into the body at the desired location at or near the flow control organ to be restored. The hydrogel may be dehydrated prior to insertion. When carrier 20 consisting of hydrogel is inserted into the body, the hydrogel will hydrate in situ.

Once biological agent 22 is associated (18 in FIG. 2), the combination is then associated (24) with tissue in the region of the body at or near the flow control organ to be restored. The combination may be physically placed at the desired site by any known and commonly available insertion technique as, for example, by use of an endoscope. Of course, it will be recognized and understood that will an endoscope may be the tool of choice for certain flow control organs such as an esophageal sphincter, that other well known medical tools may be utilized for these organs as well as other flow control organs located outside of the upper gastrointestinal tract.

Biological agent 22 may be placed into tissue at the desired region by conventional means. In a preferred embodiment, biological agent 22 is placed in muscle tissue in such region.

The contents of co-pending application entitled “Method and Article to Restore Function to GI or Urinary Tract in a Patient” by inventor Warren L. Starkebaum (Attorney Docket No. 151P21313USO1), filed on even date herewith, is hereby incorporated by reference in its entirety.

Thus, embodiments of the method and article to restore function to a flow control location of a patient are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A method of restoring function to a flow control location in a patient, comprising the steps of:
inserting a biological agent into a region near said flow control location; and
stabilizing said biological agent in said region;
wherein said biological agent comprises a biological agent selected from a group consisting of growth factors, fibroblast cells, smooth muscle cells, endothelial cells, stem cells and combinations thereof.

2. A method as in claim 1 wherein said inserting step comprises the step of injecting said biological agent into tissue in said region.

3. A method as in claim 1 wherein said biological agent is associated with a carrier.

4. A method as in claim 3 wherein said carrier comprises a polymer matrix.

5. A method as in claim 4 wherein said polymer matrix comprises a non-biodegradable polymer matrix.

6. A method as in claim 4 wherein said polymer matrix comprises a biodegradable polymer matrix.

7. A method as in claim 3 wherein said carrier comprises a hydrogel.

8. A method as in claim 3 wherein said carrier comprises a dehydrated hydrogel.

9. A method as in claim 7 further comprising the step of allowing said hydrogel to hydrate.

10. A method as in claim 1 wherein said region is selected from a group consisting of a gastro-intestinal location, a urinary location and a vascular location.

11. A method as in claim 10 wherein said region comprises a location selected from the group consisting of an upper esophageal sphincter, lower esophageal sphincter, pylorus, urinary sphincter and anal sphincter.

12. A method as in claim 1 wherein said stabilizing step comprises placing said biological agent into tissue near said region.

13. A method as in claim 12 wherein said tissue comprises muscle tissue.

14. A method as in claim 1 wherein said stabilizing step comprises inserting a carrier containing biological agent into said region.

15. A method as in claim 14 wherein said stabilizing step further comprises inserting said carrier into tissue located at said region.

16. A method as in claim 15 wherein said tissue comprises muscle tissue.

17. A method as in claim 14 wherein said carrier comprises a non-biodegradable polymer matrix.

18. A method as in claim 14 wherein said carrier comprises a hydrogel.

19. A method as in claim 18 wherein said carrier comprises a dehydrated hydrogel.

20. An article intended to restore function to a flow control location in a patient, comprising:
a carrier adapted to be inserted into a region near said flow control location; and
a biological agent, associated with said carrier, selected from a group consisting of growth factors, fibroblast cells, smooth muscle cells, endothelial cells, stem cells and combinations thereof.

21. An article as in claim 20 wherein said carrier is adapted to be inserted into tissue in said region.

22. An article as in claim 21 wherein said tissue comprises muscle tissue.

23. An article as in claim 20 wherein said carrier comprises a polymer matrix.

24. An article as in claim 23 wherein said polymer matrix comprises a non-biodegradable polymer matrix.

25. An article as in claim 23 wherein said polymer matrix comprises a biodegradable polymer matrix.

26. An article as in claim 20 wherein said carrier comprises a hydrogel.

27. An article as in claim 20 wherein said carrier comprises a dehydrated hydrogel.

28. An article as in claim 20 wherein region comprises a location selected from the group of an upper esophageal sphincter, lower esophageal sphincter, pylorus, urinary sphincter and anal sphincter.
